(19) United States
(12) Patent Application Publication
(10) Pub. No.: US 2022/0411882 A1
(43) Pub. Date: Dec. 29, 2022

(54) SNP MOLECULAR MARKER FOR WEIGHT GAIN TRAIT SELECTION AND GENETIC SEX IDENTIFICATION OF ICTALURUS PUNCTATUS AS WELL AS SCREENING METHOD AND APPLICATION OF SNP MOLECULAR MARKER

(71) Applicant: Freshwater Fisheries Research Institute of Jiangsu Province, Nanjing (CN)

(72) Inventors: Shiyong Zhang, Nanjing (CN); Xiaohui Chen, Nanjing (CN); Minghua Wang, Nanjing (CN); Liqiang Zhong, Nanjing (CN); Siqi Xu, Nanjing (CN); Yongqiang Duan, Nanjing (CN); Hongyan Liu, Nanjing (CN); Junjie Shao, Nanjing (CN); Wenji Bian, Nanjing (CN)

(73) Assignee: Freshwater Fisheries Research Institute of Jiangsu Province, Nanjing (CN)

(21) Appl. No.: 17/845,893

(22) Filed: Jun. 21, 2022

(30) Foreign Application Priority Data
Jun. 21, 2021 (CN) 202110687624.5
Jun. 21, 2021 (CN) 202110688122.4
Jul. 14, 2021 (CN) 202110796789.6

(51) Int. Cl. C12Q 1/6888; C12Q 1/6879

(52) U.S. Cl. CPC C12Q 1/6888; C12Q 1/6879; C12Q 2600/124; C12Q 2600/156; C12Q 1/6858

(57) ABSTRACT

An SNP molecular marker for weight gain trait selection and genetic sex identification of Ictalurus punctatus as well as a screening method and application of the SNP molecular marker are provided. At least one of 17 SNP molecular markers for weight gain trait selection of Ictalurus punctatus and a molecular control means for genetic sex and weight gain trait selection and control of Ictalurus punctatus are further provided. Efficient and scientific identification of Ictalurus punctatus is achieved by means of simple PCR reactions and nucleic acid test strips, and the accuracy rate reaches 100%. A traditional agarose gel electrophoresis method is not used in the whole identification process, the cumbersome steps of gel preparation and running electrophoresis are omitted, nucleic acid dyes are not used, and the experimental process is safe and environmentally friendly.

### CROSS REFERENCE TO THE RELATED APPLICATIONS

This application is based upon and claims priority to Chinese Patent Applications No.: 202110796789.6, filed on Jul. 14, 2021, No.: 202110688122.4, filed on Jun. 21, 2021, and No.: 202110687624.5, filed on Jun. 21, 2021, the entire contents of which are incorporated hereby by reference.

### SEQUENCE LISTING

The instant application contains a Sequence Listing which has been submitted in ASCII format via EFS-Web and is hereby incorporated by reference in its entirety. Said ASCII copy is named GBLB042_SequenceListing.txt, created on Jun. 21, 2022, and is 14,374 bytes in size.

### TECHNICAL FIELD

The present disclosure belongs to the technical field of molecular marker selection of fish growth traits, and relates to an SNP (Single Nucleotide Polymorphism) molecular marker for weight gain trait selection and genetic sex identification of Ictalurus punctatus as well as a screening method and application of the SNP molecular marker.

### BACKGROUND

Ictalurus punctatus, belonging to Ictaluridae, Siluriformes, is native to North America, is the freshwater fish breed having the most mature breeding technology and the highest yield in the United States, and has the advantages of strong adaptability to the environment, high muscle quality, easiness in processing and the like. With the improvement of the high-throughput sequencing technology and the reduction of the cost, GWAS research based on genome sequencing is gradually applied to genetic analysis of various traits of animals and plants. The application of GWAS in the breeding field was first carried out in cows, and Daetwyler et al. (2008) and Pryce et al. (2010) successively screened out SNP sites, associated with traits such as lactation and lactation sustainability, in cow genomes. In the field of genomics of aquatic animals, the GWAS research method has developed into one of the hottest research directions in recent years, which provides infinite possibilities for further analyzing the genetic basis for controlling complex quantitative traits of the aquatic animals and mining potential functional genes and molecular markers for breeding.

### SUMMARY

In order to overcome problems in the prior art, the present disclosure provides an SNP molecular marker for weight gain trait selection and genetic sex identification of Ictalurus punctatus as well as a screening method and application of the SNP molecular marker.

In order to achieve the above purpose, the present disclosure is achieved through the following technical scheme: the SNP molecular marker for weight gain trait selection of Ictalurus punctatus, comprising at least one of following SNP molecular markers, wherein

a first SNP molecular marker for weight gain trait selection is located at base 14,657,971 of chromosome 20, the mutation type is C/G, the molecular marker is named g. 20.14657971 C>T, and the nucleotide sequence of the molecular marker is shown in SEQ ID NO: 69; and
a second SNP molecular marker for weight gain trait selection is located at base 14,658,012 of chromosome 20, the mutation type is C/T, the molecular marker is named g. 20.14658012 C>G, and the nucleotide sequence of the molecular marker is shown in SEQ ID NO: 62;
a first sex-linked SNP molecular marker based on zbtb38-Y is located at base 366 of a zbtb38-Y gene coding region, the mutation type is G/A, the molecular marker is named g.zbtb38ycds 366 G>A, and the nucleotide sequence of the molecular marker is shown in SEQ ID NO: 47 in a sequence table;
a second sex-linked SNP molecular marker based on zbtb38-Y is located at base 431 of the zbtb38-Y gene coding region, the mutation type is G/A, the molecular marker is named g.zbtb38ycds 431 G>A, and the nucleotide sequence of the molecular marker is shown in SEQ ID NO: 48 in the sequence table;
a third sex-linked SNP molecular marker based on zbtb38-Y is located at base 733 of the zbtb38-Y gene coding region, the mutation type is G/A, the molecular marker is named g.zbtb38ycds 733 G>A, and the nucleotide sequence of the molecular marker is shown in SEQ ID NO: 49 in the sequence table;
a fourth sex-linked SNP molecular marker based on zbtb38-Y is located at base 953 of the zbtb38-Y gene coding region, the mutation type is A/C, the molecular marker is named g.zbtb38ycds 953 A>C, and the nucleotide sequence of the molecular marker is shown in SEQ ID NO: 50 in the sequence table;
a fifth sex-linked SNP molecular marker based on zbtb38-Y is located at base 1,102 of the zbtb38-Y gene coding region, the mutation type is G/A, the molecular marker is named g.zbtb38ycds 1102 G>A, and the nucleotide sequence of the molecular marker is shown in SEQ ID NO: 51 in the sequence table;
a sixth sex-linked SNP molecular marker based on zbtb38-Y is located at base 1,107 of the zbtb38-Y gene coding region, the mutation type is T/A, the molecular marker is named g.zbtb38ycds 1107 T>A, and the nucleotide sequence of the molecular marker is shown in SEQ ID NO: 52 in the sequence table;
a seventh sex-linked SNP molecular marker based on zbtb38-Y is located at base 2,073 of the zbtb38-Y gene coding region, the mutation type is T/C, the molecular marker is named g.zbtb38ycds 2073 T>C, and the nucleotide sequence of the molecular marker is shown in SEQ ID NO: 53 in the sequence table;
an eighth sex-linked SNP molecular marker based on zbtb38-Y is located at base 2,409 of the zbtb38-Y gene coding region, the mutation type is T/C, the molecular marker is named g.zbtb38ycds 2409 T>C, and the nucleotide sequence of the molecular marker is shown in SEQ ID NO: 54 in the sequence table;
a ninth sex-linked SNP molecular marker based on zbtb38-Y is located at base 2,825 of the zbtb38-Y gene coding region, the mutation type is T/C, the molecular marker is named g.zbtb38ycds 2825 T>C, and the nucleotide sequence of the molecular marker is shown in SEQ ID NO: 55 in the sequence table;
a tenth sex-linked SNP molecular marker based on zbtb38-Y is located at base 2,836 of the zbtb38-Y gene coding region, the mutation type is C/T, the molecular marker is named g.zbtb38ycds 2836 C>T, and the nucleotide sequence of the molecular marker is shown in SEQ ID NO: 56 in the sequence table;
an eleventh sex-linked SNP molecular marker based on zbtb38-Y is located at base 3,070 of the zbtb38-Y gene coding region, the mutation type is A/G, the molecular marker is named g.zbtb38ycds 3070 A>G, and the nucleotide sequence of the molecular marker is shown in SEQ ID NO: 57 in the sequence table;
a twelfth sex-linked SNP molecular marker based on zbtb38-Y is located at base 3,177 of the zbtb38-Y gene coding region, the mutation type is T/C, the molecular marker is named g.zbtb38ycds 3177 T>C, and the nucleotide sequence of the molecular marker is shown in SEQ ID NO: 58 in the sequence table; and
a thirteenth sex-linked SNP molecular marker based on zbtb38-Y is located at base 3,375 of the zbtb38-Y gene coding region, the mutation type is A/G, the molecular marker is named g.zbtb38ycds 3375 A>G, and the nucleotide sequence of the molecular marker is shown in SEQ ID NO: 59 in the sequence table.

As one of the aspects of the present disclosure, the present disclosure provides the screening method of an SNP molecular marker for weight gain trait selection of Ictalurus punctatus, comprising the following steps: 1) extracting DNA of fish samples to be tested, constructing genome re-sequencing libraries of Ictalurus punctatus, and performing PE150 paired-end sequencing on all the libraries on a BGISEQ-500 sequencing platform; 2) obtaining the monthly weight gain of the fish samples to be tested; 3) performing sequencing data filtering and SNP typing: after filtering and quality control, original sequencing data are compared with a reference genome of Ictalurus punctatus, and SNPs are detected by means of GATK software, captured variations are annotated by means of ANNOVAR software, and genetic locations of the genetic variations are determined by means of VCFtools software; and 4) performing genome-wide association study of growth traits: GWAS (genome-wide association study) of the weight gain traits is performed by means of a single site mixed linear model GWAS (EMMAX) of SNP & Variation Suit v8.5.0 software, and thus the target SNP molecular marker is obtained.

As one of the aspects of the present disclosure, the present disclosure provides the method for rapidly detecting genetic sexes of Ictalurus punctatus with an SNP molecular marker, comprising the following steps: 1) designing peripheral primers, specific extension primers and DNA probes for amplifying SNP sites for genetic sex selection of Ictalurus punctatus, wherein the sequences of the peripheral primers, the ASE primers and the DNA probes are shown in SEQ ID NO: 63 to SEQ ID NO: 66, and last bases at 3′ ends of the ASE primers are male-specific bases of Ictalurus punctatus; 2) marking 5′ ends of the ASE primers with FAM fluorophores, marking 3′ ends of probes ASE-DP with Biotin, and testing the relationship between secondary structures of the ASE primers and the probes ASE-DP by means of Oligo software, so as to avoid primer dimers; 3) performing PCR (polymerase chain reaction) amplification by means of the DNA of the fish samples to be tested and the peripheral primers in step 1) to obtain first-round PCR amplification products; 4) performing second-round PCR extension reaction and nucleic acid hybridization by means of the first-round amplification products in step 3) and the ASE primers and the probes ASE-DP in step 1) to obtain second-round PCR amplification products and ASE-DP hybridization nucleic acid products; and 5) detecting the hybridization nucleic acid products by means of rapid nucleic acid test strips, and judging the genetic sexes of the fish samples to be tested according to color development results of the test strips.

As one of the aspects of the present disclosure, the present disclosure provides a method for developing sex-linked SNP molecular markers of Ictalurus punctatus on the basis of male-specific genes zbtb38-Y of Ictalurus punctatus, comprising the following steps: 1) designing specific primers for zbtb38 gene coding regions of Ictalurus punctatus, wherein the sequences of the specific primers are shown in SEQ ID NO: 1 to SEQ ID NO: 44; 2) performing first-round PCR amplification by means of genomic DNA of female and male Ictalurus punctatus and the specific primers to obtain first-round products; 3) performing second-round PCR amplification by means of the first-round products in step 2) and a barcode to obtain second-round products; 4) constructing sequencing libraries after mixing the second-round female and male amplification products in step 3), wherein the sequencing libraries are subjected to 150 bp paired-end sequencing on an Illumina HiSeqX-ten sequencing platform; and 5) comparing original sequencing data with zbtb38 genes after filtering the original sequencing data, and regarding SNP molecular markers, linked with sexes, in samples with a comparison rate greater than 80% as sex-linked SNP molecular markers.

As one of the aspects of the present disclosure, the present disclosure provides the application of an SNP molecular marker to genetic sex identification, comprising the following steps: 1) designing male-specific amplification primers for sex-linked SNP molecular markers, wherein in the male-specific amplification primers, the first to last base (g.zbtb38ycds 1107 T>A) and the sixth to last base (g.zbtb38ycds 1102 G>A) of a forward primer sequence are male-specific bases, and the first to last base (g.zbtb38ycds 2073 T>C) of a reverse primer sequence is a male-specific base;

2) designing a pair of control primers in a region without mutation sites, wherein the control primers may amplify bands in both female and male individuals; and 3) establishing double PCR amplification reaction by means of the male-specific amplification primers and the control primers, and detecting PCR amplification products by means of 1% agarose gel.

Preferably, the sequences of the male-specific amplification primers are shown in SEQ ID NO: 45 and SEQ ID NO: 46.

As one of the aspects of the present disclosure, the present disclosure provides the primer for developing the SNP molecular marker for trait selection of Ictalurus punctatus and the kit of the primer, wherein the primer comprises one or more of sequences shown in SEQ ID NO: 1 to SEQ ID NO: 59 and SEQ ID NO: 61 to SEQ ID NO: 66.

The present disclosure has the beneficial effects as follows: the present disclosure provides the SNP molecular marker for trait selection of Ictalurus punctatus and the application, discloses at least one of 17 SNP molecular markers for trait selection of Ictalurus punctatus, and provides a molecular control means for genetic sex and weight gain trait selection and control of Ictalurus punctatus. Efficient and scientific identification of Ictalurus punctatus is achieved by means of simple PCR reactions and nucleic acid test strips, and the accuracy rate reaches 100%. A traditional agarose gel electrophoresis method is not used in the whole identification process, the cumbersome steps of gel preparation and running electrophoresis are omitted, nucleic acid dyes are not used, and the experimental process is safe and environmentally friendly.

### BRIEF DESCRIPTION OF THE DRAWINGS

FIG. 1 shows monthly weight gain statistics of Ictalurus punctatus of three genotypes in Embodiment 1, note: **p<0.01; *p<0.05;

FIG. 2 shows detection results of female individuals of Ictalurus punctatus in Embodiment 3;

FIG. 3 shows detection results of male individuals of Ictalurus punctatus in Embodiment 3;

FIG. 4 shows color development conditions of nucleic acid test strips for Ictalurus punctatus in Embodiment 3, wherein the first test strip shows the color development condition of the nucleic acid test strip for Ictalurus punctatus of the female genetic sex, and the second test strip shows the color development condition of the nucleic acid test strip for Ictalurus punctatus of the male genetic sex; \textcircled{1} refers to a quality inspection line, \textcircled{2} refers to a test line, and \textcircled{3} refers to a quality inspection line;

FIG. 5 shows linkage analysis results of 16 SNPs in a zbtb38 gene coding region of Ictalurus punctatus in Embodiment 4;

FIG. 6 is a sequencing peak diagram of 13 sex-linked SNPs in a zbtb38 gene coding region of Ictalurus punctatus in Embodiment 4;

FIG. 7 shows six amino acid variations of protein encoded by zbtb38 genes on X and Y chromosomes of Ictalurus punctatus in Embodiment 4;

FIG. 8 shows results of genetic sex identification of Ictalurus punctatus on the basis of SNP molecular markers in Embodiment 5.

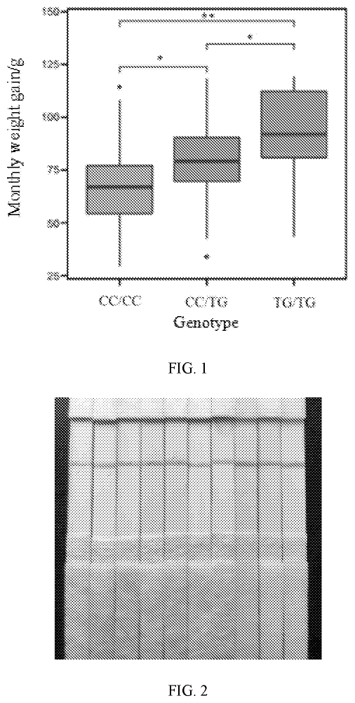

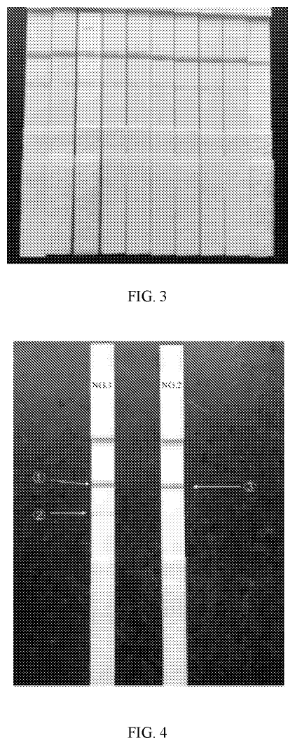

### DETAILED DESCRIPTION OF THE EMBODIMENTS

Embodiment 1: screening of SNP molecular markers associated with weight gain traits of Ictalurus punctatus

1. A breeding experiment was performed as follows: after 180 days of independent breeding of juvenile fishes from different Ictalurus punctatus families, 50 juvenile fishes of equivalent sizes were selected from each family, weight data of the juvenile fishes were recorded, and a PIT electronic tag with a 12-digit identification code was injected into the abdominal cavity of each fish. After wounds were completely healed, all the experimental fishes were put into a pond of 0.2 hm² for mixed breeding. A feeding principle of “fixed time, fixed point and quantification” was strictly implemented in the breeding process.

2. Experimental sample collection was performed as follows: after one year of breeding, the experimental fishes were captured, weight data of each fish were recorded again, and meanwhile, tissue of tail fins of experimental Ictalurus punctatus was collected for DNA extraction. The monthly weight gain of each fish was calculated according to the weight data before and after breeding.

3. Extraction and detection of genomic DNA were performed as follows: genomic DNA was extracted from tail fins of the experimental fishes to prepare genome-wide DNA samples of Ictalurus punctatus. Quality control was performed on the samples by means of a Qubit and 1% agarose gel electrophoresis respectively. The qualified DNA (with the total amount larger than 3 μg, the concentration higher than 30 ng/μL and the ratio of OD260/OD280 in the range of 1.80-2.00) was used for construction of sequencing libraries in the next step.

4. Construction and sequencing of libraries were performed as follows: the genomic DNA of each sample was broken into DNA fragments of 50 bp to 800 bp by means of Covaris E220 (Covaris, Brighton, UK) ultrasonic waves. DNA fragments of 100 bp to 300 bp were further fished by means of an Agencourt AMPure XP bead kit (Beckman, Krefeld, Germany). Then ends of the fished DNA fragments were repaired, and dATP (deoxyadenosine triphosphate) was added to 3′ ends to obtain sticky ends. Adapter sequences with dTTP (deoxy-thymidine triphosphate) tails were connected to both ends of DNA fragments of each sample to distinguish the various samples. Then single-stranded circular DNA was amplified for 8 cycles through a rolling circle amplification technology. Single strand cyclization treatment was carried out in the following steps: a PCR product and a special molecule were thermally denatured together, the special molecule was inversely complementary to a special strand of the PCR product, and a single-stranded molecule was connected through DNA ligase. Remaining linear molecules were digested with exonuclease to obtain the single-stranded circular DNA finally. 303 samples were mixed finally to construct 19 sequencing libraries. PE150 paired-end sequencing was performed on all the libraries on the BGISEQ-500 sequencing platform.

5. Sequencing data filtering and SNP typing were performed as follows: bioinformatics analysis was started after original data generated by the BGISEQ-500 sequencing platform were offline. In order to improve the sequence quality, the original data were filtered by means of Trimmomatic (version 0.36) software. Forward and reverse FASTQ sequences of each sample were used as input files, and parameters of the software were “LEADING:3 TRAILING:3 SLIDINGWINDOW:4:15 MINLEN:75”. High-quality reads obtained were used for subsequent analysis. Genome-wide SNP variations were identified in clean reads of the 303 samples. The high-quality clean reads were compared with a reference genome sequence of Ictalurus punctatus by means of BWA software (bwa mem -k 32). Files in the SAM format were imported into SAMtools software for sequencing and merging, and duplicate reads were deleted in combination with Picard software. SNPs were detected on valid BAM files by means of a “UnifiedGenotyper” module of GATK software (version 2.4-9). According to a gene annotation file of a reference genome, the captured variations were annotated by means of ANNOVAR software. Finally, the genetic locations of all the variations were determined by means of the VCFtools software (version 0.1.10).

6. GWAS of the growth traits was performed as follows: GWAS of the growth traits was performed by means of the single site mixed linear model GWAS (EMMAX) of SNP & Variation Suit v8.5.0 software. An EMMAX (Efficient Mixed Model Association expedited) statistical test method was superior to principal component analysis in the aspect of explaining sample structures (stratification and correlation). In this study, this technology was used to correct a hybrid effect caused by correlations among subgroup structures and individuals. In EMMAX, nxn genetic relation matrices among the individuals were calculated according to genotype data. The model used was y=Xβ+Zu+e, wherein y was an n×1 vector of a phenotypic value, X was an n×f matrix of a fixed effect including the SNPs and the age (month), β was a q×1 vector representing a fixed effect coefficient, Z was an n×t matrix associated with a random effect, the phenotypic value and the β fixed effect coefficient, u was a random animal effect, and e was a residual effect.

Result analysis was as follows: 3.69 Tb of original data were produced in total through sequencing, with an average of 12.18 Gb for each sample. Through filtering and counting of the original offline data, 3.64 Tb of clean data were produced after barcode sequences of sequencing adapters and low-quality sequencing data were removed, with an average of 12.01 Gb (6.8-26.20 Gb) for each sample, and the average values of Q20 and Q30 were 97.81% and 93.05% respectively. The clean reads were compared with the reference genome of Ictalurus punctatus, the average mapping rate was 94.85%, and the range was 90.05%-96.99%. Results of filtering component statistics of the original data, base mass distribution of the clean reads and base content of the clean reads indicated that the quality of various items of genome re-sequencing this time was high and subsequent analysis could be met. The clean reads of each sample were compared with the reference genome sequence, 12,045,859 original SNPs were obtained in total, and 5,641,711 SNPs shared by all samples were finally screened out from the original SNPs.

GWAS was performed on 5,641,711 SNP molecular markers in the whole genome and the monthly weight gain by means of the EMMAX software. 15 markers were associated with the monthly weight gain (MWG) trait (P≤1×10⁻⁶), wherein the two SNPs located on locations 14,657,971 and 14,658,012 of chromosome 20 were extremely significantly associated with the monthly weight gain (P≤1×10⁻⁸) (FIG. 1), and the two SNPs were located in intron 9 of the rrp44 gene. The mutation type of the first SNP was C/T, the SNP was named g. 20.14657971 C>T, and the nucleotide sequence of the SNP was shown in SEQ ID NO: 69 (TACTAACTAGSCTCTTTAAAA); and the mutation type of the second SNP was C/G, the SNP was named g. 20.14658012 C>G, and the nucleotide sequence of the SNP was shown in SEQ ID NO: 62 (GTTGAAAACTYTGATTTTTAC), which resulted in polymorphisms.

TABLE 1.1
SNP Molecular Markers Associated with Weight Gain Traits
of Ictalurus punctatus (Note: *P < 1E−07)

| SNP | Chromo- | Reference | Variant | Minor Allele | |
|---|---|---|---|---|---|
| Location | some | Base | Base | Frequency | P Value |
| 14658012 | 20 | C | G | 0.20130 | 1.97E−09* |
| 14657971 | 20 | C | T | 0.19970 | 1.45E−08* |

Linkage analysis of the two SNP sites was performed by means of SHEsis software, and the result showed that the two sites were subjected to linkage inheritance, there were two haploid genotypes, namely, TG and CC, which resulted in polymorphisms. Statistical results of weight gain data of individuals of various genotypes showed that the weight gain traits of the individuals of the TG/TG genotype were extremely significantly better than those of the individuals of the CC/CC genotype (p<0.01), and were significantly better than those of the individuals of the CC/GT genotype (p<0.05). The results are shown in FIG. 1.

The screening method of the SNP molecular markers, provided in this embodiment, comprises the following steps:

1) performing experimental sample collection: tissue of tail fins of experimental Ictalurus punctatus is collected for DNA extraction; 2) performing measurement of growth data: weight data of Ictalurus punctatus before and after breeding are measured, and the monthly weight gain of each fish is calculated; 3) performing extraction and detection of genomic DNA: the genomic DNA is extracted from the tail fins of the experimental fishes to prepare genome-wide DNA samples of Ictalurus punctatus, and associated mass values are detected; 4) performing construction and sequencing of libraries: genome re-sequencing libraries of Ictalurus punctatus are constructed by means of the qualified DNA samples, and PE150 paired-end sequencing is performed on all the libraries on the BGISEQ-500 sequencing platform; and 5) performing sequencing data filtering and SNP typing: after filtering and quality control, original sequencing data are compared with a reference genome of Ictalurus punctatus. SNPs are detected by means of GATK software, captured variations are annotated by means of ANNOVAR software, and genetic locations of the genetic variations are determined by means of VCFtools software; and 6) performing GWAS of growth traits: GWAS of the weight gain traits is performed by means of the single site mixed linear model GWAS (EMMAX) of the SNP & Variation Suit v8.5.0 software, and the SNP molecular markers extremely significantly associated are selected. In order to verify the reliability of the two mined SNP molecular markers, weight gain testing and SNP typing based on Sanger sequencing are performed on another Ictalurus punctatus breeding group, which further confirms that the polymorphisms of the two SNP sites are extremely significantly associated with the weight gain traits of Ictalurus punctatus (p<0.01). Due to linkage inheritance of the two SNP markers, one of the SNP markers can be used to select and improve the weight gain traits of Ictalurus punctatus.

Embodiment 2: verification of SNP molecular markers associated with weight gain traits of Ictalurus punctatus

1. A breeding experiment and sample collection were performed as follows: after Ictalurus punctatus fries started eating, the Ictalurus punctatus fries from different families were bred in a mixed mode in a pond of 3 mu for 18 months. The breeding mode was the same as that in Embodiment 1. 201 fishes were randomly selected and weighed, and tissue of tail fins was collected.

2. Extraction and detection of genomic DNA were performed as follows: the genomic DNA was extracted from the tail fins of the experimental fishes, quality control was performed on the samples by means of a Qubit ultraviolet spectrophotometer and 1% agarose gel electrophoresis respectively, and the qualified DNA (with the concentration higher than 30 ng/μL and the ratio of OD260/OD280 in the range of 1.80-2.00) was used for SNP typing in the next step.

3. SNP typing was performed as follows: primers (SEQ ID NO: 67, SEQ ID NO: 68) for simultaneously amplifying two SNP sites (g. 20.14657971 C>T and g. 20.14658012 C>G) were designed by a Primer BLAST function in NCBI. A 2× Taq Plus Master Mix (Dye Plus) kit (Vazyme Biotech Co., Ltd) was used for PCR amplification, and had a reaction system of 2× Taq Master Mix of 10 μL, genomic DNA of 1 μL, each 10 μM primer of 0.5 μL, and ddH2O of 7 μL. Amplification conditions included pre-denaturation at 94° C. for 5 min, denaturation at 94° C. for 30 s, annealing at 55° C. for 30 s, extension at 72° C. for 30 s, 30 cycles and extension at 72° C. for 10 min. The PCR amplification products were subjected to electrophoresis by means of 1% agarose gel. After electrophoresis, the gel was placed on an UV gel detector to observe electrophoresis results and determine the number and size of amplified bands of each sample. The qualified PCR amplification products were subjected to bi-directional Sanger sequencing by means of an ABI 3730 sequencer, and primers used for sequencing were the same as those used for PCR amplification. A Sanger sequencing peak diagram was read by means of Chromas software, and the genotypes of each sample at two SNP sites (g. 20.14657971 C>T and g. 20.14658012 C>G) were recorded.

4. Weight trait and SNP association study was performed as follows: after successful typing of each sample at the two SNP sites (g. 20.14657971 C>T and g. 20.14658012 C>G), association study was performed on weight traits and the genotypes of the samples. Results were shown in Table 2.1 and showed that the weight of individuals of the TG/TG genotype was extremely significantly higher than that of individuals of the CC/CC genotype (p<0.01), and was significantly higher than that of individuals of the CC/GT genotype (p<0.05). The haploid homozygous genotype TG/TG had the weight gain advantage.

TABLE 2.1
Association Study Results of Weight Traits
of Ictalurus punctatus and Three Genotypes

| Genotype | Weight (g) |
|---|---|
| CC/CC | 943.28 ± 266.29^{a} |
| CC/TG | 983.95 ± 244.94^{a} |
| TG/TG | 1083.06 ± 306.77^{b} |

According to Embodiments 1 and 2, the two SNP molecular markers associated with the weight gain traits were screened out from chromosome 20 of Ictalurus punctatus for the first time through genome-wide association study. The BGISEQ-500 platform was mainly used to carry out genome-wide re-sequencing on Ictalurus punctatus breeding groups which came from different families and had equivalent initial body mass, and association study was carried out between the genome-wide SNP variations and the weight gain traits to mine the important SNP molecular markers controlling the weight gain traits. The correlation between the two SNP molecular markers and the weight gain traits of Ictalurus punctatus was further verified in another breeding group by means of the genotyping technology based on Sanger sequencing. In order to reduce the influence of environmental factors on the experimental results, all the offspring of the families were bred in the same environment, and the test fishes had similar specifications. The obtained SNP markers could be used for molecular marker assisted breeding of Ictalurus punctatus, and the individuals with the weight gain advantage were selected as parents for fry breeding.

Embodiment 3: a method for rapidly detecting genetic sexes on the basis of extension reactions of specific primers of sex-linked SNP sites of Ictalurus punctatus

96 individuals of Ictalurus punctatus were randomly caught from a breeding pond, and tail fins of Ictalurus punctatus were taken and placed in EP tubes of 1.5 mL to be preserved with absolute ethanol. Among the 96 Ictalurus punctatus individuals whose genetic sexes had been determined, 10 samples of tail fin tissue of the individuals with the genetic sex being male and 10 samples of tail fin tissue of the individuals with the genetic sex being female were randomly selected for testing.

### 1. Extraction of DNA of Ictalurus punctatus

1.1 Digestion and lysis of the tail fins of Ictalurus punctatus were performed as follows: 20 mg of the preserved tail fin tissue was taken, cut into pieces and then placed into the EP tubes of 1.5 mL. DNA was extracted by means of a FastPureCell/Tissue DNA Isolation Mini Kit (Vazyme Biotech Co., Ltd). 230 μL of a Buffer GA and 20 μL of PK protein working fluid were added and were subjected to vortex mixing for 15 s, and the tubes were placed in a water bath at 55° C. for overnight digestion. 250 μL of a Buffer GB was added to digestive fluid after complete enzymatic hydrolysis of the tissue and was subjected to vortex mixing for 20 s and water bath treatment at 70° C. for 10 min.

1.2 Column chromatography of the digestive fluid was performed as follows: 180 μL of absolute ethanol was added to the digestive fluid and was subjected to vortex mixing for 15-20 s. Adsorption columns (gDNA columns) were placed in collection tubes of 2 mL, the mixture obtained in the previous step was transferred to the adsorption columns, and centrifugation at 12,000 rpm (13,400×g) was performed for 1 min. Filtrate was discarded, and the adsorption columns were placed in the collection tubes. 500 μL of a Washing Buffer A was added to the adsorption columns. Centrifugation at 12,000 rpm was performed for 1 min. Filtrate was discarded, the adsorption columns were placed in the collection tubes, 650 μL of a Washing Buffer B was added to the adsorption columns, and centrifugation at 12,000 rpm was performed for 1 min. The previous step was repeated. Filtrate was discarded, and the adsorption columns were placed in the collection tubes. Empty tube centrifugation at 12,000 rpm was performed for 2 min. The adsorption columns were placed in new centrifuge tubes of 1.5 ml. 50 μL of an Elution Buffer preheated to 70° C. was added to centers of membranes of the adsorption columns and was placed at the room temperature for 3 min. Centrifugation at 12,000 rpm (13,400×g) was performed for 1 min. The adsorption columns were discarded, and DNA was placed at subzero 20° C. for long-term preservation.

### 2. Screening of SNP Sites for Genetic Sex Identification of Ictalurus punctatus and Design of Primers

The sex-linked SNP site g.zbtb38ycds 366 G>A was selected as an SNP site for rapid genetic sex identification of Ictalurus punctatus, the site was a site on zbtb38-Y. Peripheral primers (WF/R), allele-specific extension (ASE) primer and DNA probes (ASE-DP) for amplifying the interval of the SNP site were designed by means of the Primer BLAST online software in NCBI, last bases at 3′ ends of the ASE primers were male-specific bases of Ictalurus punctatus, 5′ ends of the ASE primers were marked with FAM fluorophores, 3′ ends of the probes ASE-DP were marked with Biotin, and the relationship between secondary structures of the ASE primers and the DNA probes was tested by means of Oligo software, so as to avoid primer dimers. The sequences of the primers were shown in Table 3.1.

TABLE 3.1
Specific Extension Reaction Primers of Sex-linked
SNP Sites of Ictalurus punctatus
(SEQ ID NO: 63 to SEQ ID NO: 66 in sequence)

| Name of Primer | Sequence of Primer | Marker |
|---|---|---|
| WF | GAAATGTCTTGGCTGCGACT | None |
| WR | CTCTTGCTCCTCAAGCCGTG | None |
| ASE | TCCCCTTTCTTGAGAAA | 5′FAM |
| ASE-DP | GCCTTTCAATCTCAAG | 3′Biotin |

Note: WF refers to a peripheral forward primer; WR
refers to a peripheral reverseprimer; ASErefers to a
specific forward primer; and ASE-DPrefers to a DNA probe.

### 3. Amplification of Sex-Linked SNP Sites of Ictalurus punctatus

3.1 An extension reaction of the peripheral primers of the sex-linked SNP sites of Ictalurus punctatus was performed as follows: 40 μL of a reaction system was used, including 20 μL of a 2× Phanta Max Master Mix (Vazyme Biotech Co., Ltd), 1 μL of a template, 1 μL of each of the peripheral primers (WF and WR) and 17 μL of H2O. A PCR amplification procedure (first-round PCR amplification reaction) was performed as follows: pre-denaturation was performed at 95° C. for 5 min; denaturation was performed at 94° C. for 30 s, annealing was performed at 57° C. to 53° C. (1° C. lowered each cycle) for 30 s, extension was performed at 72° C. for 60 s, and 2 cycles were performed at each annealing temperature; denaturation was performed at 94° C. for 30 s, annealing was performed at 55° C. for 30 s, extension was performed at 72° C. for 60 s, and 15 cycles were performed; and extension was performed at 72° C. for 10 min.

3.2 Extension reactions of specific primers of sex-linked SNP sites of Ictalurus punctatus were performed as follows: first-round PCR amplification reaction products were diluted 10 times and used as a template of a second-round PCR amplification reaction. 20 μL of a reaction system was used, including 10 μL of a 2× Phanta Max Master Mix (Vazyme Biotech Co., Ltd), 1 μL of the template, 1 μL of each of the ASE primer (ASE-F) and the DNA probe (ASE-DP), and 7 μL of H2O. A PCR amplification procedure (second-round PCR amplification reaction) was performed as follows: treatment was performed at 95° C. for 5 min; treatment was performed at 94° C. for 30 s; treatment was performed at 66° C. for 30 s, and 20 cycles were performed; treatment was performed at 94° C. for 30 s; treatment was performed at 50° C. for 30 s, and 25 cycles were performed. Second-round PCR amplification products obtained finally and hybridization nucleic acid products of the DNA probe were preserved at subzero 20° C.

4. Genetic Sex Identification of Ictalurus punctatus

4.1 Detection of amplification products was performed as follows: 20 μL of a buffer (USTAR BIOTECHNOLOGIES (HANGZHOU) LTD) used in cooperation with rapid nucleic acid test strips was dropped in a 100 μL microporous plate, 10 μL of the second-round PCR amplification products and hybridization nucleic acid products of the DNA probe were taken and dropped on sample pads of the rapid nucleic acid test strips. The nucleic acid test strips containing the hybridization nucleic acid products were inserted into the 100 μL microporous plate containing the buffer, and it was ensured that lowest ends of the sample pads made contact with the buffer.

4.2 Determination of results was performed as follows: the results were read within 10-15 min after the process was completed. Red bands were observed in reading regions of the rapid nucleic acid test strips. During reading of the 20 sample results, the red bands appeared in quality control regions of the reading regions of the rapid nucleic acid test strips, and red bands appeared in detection regions of the DNA samples of 10 male individuals of Ictalurus punctatus; however, no red bands were found in detection regions of the DNA samples of 10 female individuals of Ictalurus punctatus.

5. Verification of a genetic sex identification method was performed as follows: 96 samples were randomly selected from a sample library of a genetics and breeding center of Ictalurus punctatus for sex identification, so as to verify the accuracy of the genetic sex identification method. The second-round PCR amplification products and the hybridization nucleic acid products of the DNA probes were detected by means of the rapid nucleic acid test strips, genetic sex identification results of the selected samples were compared with sex information recorded in a database, and results showed that the accuracy of the genetic sex identification method reached 100%.

In the embodiment, the sequences of the sex-linked SNP sites were enriched through extraction of the genomic DNA of the tissue of the tail fins of Ictalurus punctatus and peripheral PCR amplification, then specific amplification of the SNP sites and hybridization of the probes were performed, SNP site detection was performed on the obtained hybridization nucleic acid products by means of the nucleic acid test strips, rapid genetic sex identification of Ictalurus punctatus was achieved by means of the simple PCR reactions and the nucleic acid test strips, efficient genetic sex identification of Ictalurus punctatus was achieved, and the accuracy reached 100%. The traditional agarose gel electrophoresis method was not used in the whole identification process, the cumbersome steps of gel preparation and running electrophoresis were omitted, the nucleic acid dyes were not used, and the experimental process was safe and environmentally friendly.

Embodiment 4: screening of sex-linked SNP markers on the basis of zbtb38 genes of Ictalurus punctatus

1. Design of primers was performed as follows: 22 pairs of specific primers were designed for the sequence of the zbtb38 gene coding region of Ictalurus punctatus to ensure that the amplification products could cross each other and cover the whole coding region. See Table 4.1 for specific primer information (SEQ ID NO: 1 to SEQ ID NO: 44 in sequence). The primers were synthesized by Sangon Biotech (Shanghai) Co., Ltd.

TABLE 4.1
Information of Multiplex PCR Amplification Primers of
zbtb38 Gene Coding Region of Ictalurus punctatus

| No. of | | | | |
|---|---|---|---|---|
| Amplicon | Sequenceinformation (5′-3′) | InitiationSite | TerminationSite | Length |
| | F: ATGATGGTGG | | | |
| | TCCATTCCAGC (SEQ ID NO: 1) | | | |
| CYH_1 | R: TCAGAGGCTG | 1 | 200 | 200 |
| | TGAAAGCGTT G | | | |
| | (SEQ ID NO: 2) | | | |
| | F: GCTGCGACTA | | | |
| | GCGGATATTT CC | | | |
| | (SEQ ID NO: 3) | | | |
| CYH_2 | R: CAGAGTCTTG | 157 | 394 | 238 |
| | CCTTTCAATC | | | |
| | TCAAG (SEQ ID NO: 4) | | | |
| | F: CAACAAGTCC | | | |
| | TTAATAGCTG | | | |
| | CAGGG (SEQ ID NO: 5) | | | |
| CYH_3 | R: TCTGTGATGG | 312 | 548 | 237 |
| | AGAAAGCATT | | | |
| | AGTTATCC (SEQ ID NO: 6) | | | |
| | F: CACCACGGCT | | | |
| | TGAGGAGC (SEQ ID NO: 7) | | | |
| CYH_4 | R: CAGCGTATGA | 482 | 694 | 213 |
| | GTGTTCAAAC | | | |
| | ATGG (SEQ ID NO: 8) | | | |
| | F: CGCTATCAGG | | | |
| | AATACCCTCT GTC | | | |
| | (SEQ ID NO: 9) | | | |
| CYH_5 | R: ACTAGTTTCA | 629 | 867 | 239 |
| | CTGGATATGG | | | |
| | GTGCC (SEQ ID NO: 10) | | | |
| | F: GACTCTGTCC | | | |
| | AACCATAGAC | | | |
| | TTGG (SEQ ID NO: 11) | | | |
| CYH_6 | R: AAGGCGGATC | 783 | 1027 | 245 |
| | ATCATCCTGT (SEQ ID NO: 12) | | | |
| | F: CCTTGTGTTC | | | |
| | TGCACCCGAC | | | |
| | (SEQ ID NO: 13) | | | |
| CYH_7 | R: GCAGTACTTG | 959 | 1170 | 212 |
| | CAAAACAGAT | | | |
| | GACTG (SEQ ID NO: 14) | | | |
| | F: CCATATGCAA | | | |
| | AGCCACAAGA | | | |
| | GAAG (SEQ ID NO: 15) | | | |
| CYH_8 | R: GGAGGATCTT | 1116 | 1340 | 225 |
| | GAAGATCAGA | | | |
| | ACTTG (SEQ ID NO: 16) | | | |
| | F: CAAGTAGCAC | | | |
| | ACCCTTCCTA | | | |
| | GAGG (SEQ ID NO: 17) | | | |
| CYH_9 | R: CTTTATTGCA | 1280 | 1525 | 246 |
| | GTAGTGACAA | | | |
| | GGATAGGC (SEQ ID NO: 18) | | | |
| | F: GACATGAGAA | | | |
| | TGTGCATTCA TGG | | | |
| | (SEQ ID NO: 19) | | | |
| CYH_10 | R: AGATTTTTTA | 1469 | 1629 | 224 |
| | TTCACTGCCA | | | |
| | GACG (SEQ ID NO: 20) | | | |
| | F: CCATCAGAAG | | | |
| | TCATTTCATG GC | | | |
| | (SEQ ID NO: 21) | | | |
| CYH_11 | F: CTGTGCCTTT | 1638 | 1876 | 239 |
| | AAGTGGAGCA GC | | | |
| | (SEQ ID NO: 22) | | | |
| | R: ACTGCCCCGC | | | |
| | TAGATGTTAA CTC | | | |
| | (SEQ ID NO: 23) | | | |
| CYH_12 | R: GTTAAAGCCT | 1828 | 2070 | 243 |
| | TCCTCTTCGA | | | |
| | TATGAGAC (SEQ ID NO: 24) | | | |
| | F: CAACATGTAG | | | |
| | AAACTTCTTT | | | |
| | GCCTTCTG (SEQ ID NO: 25) | | | |
| CYH_13 | R: GGAATGGCAT | 2005 | 2248 | 244 |
| | AACATCTCTG | | | |
| | CTTG (SEQ ID NO: 26) | | | |
| | F: ACTCATCTCC | | | |
| | CACACAATGC | | | |
| | AGTAG (SEQ ID NO: 27) | | | |
| CYH_14 | R: ACCAGGACAA | 2195 | 2430 | 236 |
| | GCAGGCTTGG | | | |
| | (SEQ ID NO: 28) | | | |
| | F: CTGACGCCGA | | | |
| | ACCTTCGC (SEQ ID NO: 29) | | | |
| CYH_15 | R: GAATCCTAGA | 2365 | 2604 | 240 |
| | TCATCCTCTG | | | |
| | CTGTGTTC (SEQ ID NO: 30) | | | |
| | F: AGAGAAGAAA | | | |
| | CTGGAAGCAA | | | |
| | GATAATG (SEQ ID NO: 31) | | | |
| CYH_16 | R: AGAGAAGAAA | 2543 | 2789 | 247 |
| | CTGGAAGCAA | | | |
| | GATAATG (SEQ IDNO: 32) | | | |
| | F: GGCAAAAGAT | | | |
| | TCAGATCCAA GC | | | |
| | (SEQ ID NO: 33) | | | |
| CYH_17 | R: GACAATATGC | 2731 | 2907 | 240 |
| | ATGCTGAGGG TG | | | |
| | (SEQ ID NO: 34) | | | |
| | F: CTGCCATTGC | | | |
| | TGTAGTAGCT CC | | | |
| | (SEQ ID NO: 35) | | | |
| CYH_18 | R: CAGTATGAGC | 2913 | 3155 | 243 |
| | AGCGGTAGCG | | | |
| | (SEQ ID NO: 36) | | | |
| | F: ACTCGATAGG | | | |
| | GTTGACTCGT | | | |
| | AGCC (SEQ ID NO: 37) | | | |
| CYH_19 | R: CTTGTGATTG | 3098 | 3341 | 244 |
| | TCTCACTATT | | | |
| | TTCTGTC (SEQ ID NO: 38) | | | |
| | F: CATGAGAGCA | | | |
| | AGCACTTCAA | | | |
| | TAAGG (SEQ ID NO: 39) | | | |
| CYH_20 | R: GTCCTGTCAC | 3283 | 3530 | 248 |
| | CTTTACAGGA | | | |
| | CAAC (SEQ ID NO: 40) | | | |
| | F: TTATCCCCAA | | | |
| | ATCCCTCAAC TCC | | | |
| | (SEQ ID NO: 41) | | | |
| CYH_21 | R: CATGTTGTAT | 33465 | 3698 | 234 |
| | AGAGGTTGGA | | | |
| | TGTGTTGAG (SEQ ID NO: 42) | | | |
| | F: GCTTTGTGGA | | | |
| | ATATTCTAAA | | | |
| | GACTCCAC (SEQ ID NO: 43) | | | |
| CYH_22 | R: TCAGTTGTGG | 3581 | 3798 | 218 |
| | GCATAAAAAA | | | |
| | CAGG (SEQ ID NO: 44) | | | |

2. Construction and sequencing of libraries

The tail fin samples of 129 Ictalurus punctatus individuals with known sexes (50 males and 79 females) were collected, and genomic DNA of the samples was extracted by means of an animal tissue DNA extraction kit (Vazyme Biotech Co., Ltd). First-round PCR was performed as follows: after synthesized primers in SEQ ID NO: 1 to SEQ ID NO: 44 were dissolved, 10 μL of each primer solution was sucked, and the Primer mix working fluid was prepared and subpackaged in a 96-pore plate. A sample plate was fully thawed and vibrated and was centrifuged at 1,000 rpm for 1 s, and then the samples were added in an automatic DNA workstation. A PCR amplification system and components are shown in Table 4.2.

A PCR procedure was performed as follows: pre-denaturation was performed at 95° C. for 15 min; denaturation was performed at 94° C. for 30 s, annealing was performed at 60° C. for 10 min, extension was performed at 72° C. for 30 s, and 4 cycles were performed; denaturation was performed at 94° C. for 30 s, annealing was performed at 60° C. for 1 min, extension was performed at 72° C. for 30 s, and 24 cycles were performed.

TABLE 4.2
Construction of First-round PCR Amplification
System of Targeted Re-sequencing Libraries

| | | Buffer | Primer mix | dNTP | UDG | Taq | | Mg²⁺ |
|---|---|---|---|---|---|---|---|---|
| | ddH₂O | 10× | 50 nM | 2.5 mM | 5 U/μL | 5 U/μL | Sample | 100 mM |
| 10 μL | 3 μL | 1 μL | 2 μL | 0.8 μL | 0.1 μL | 0.1 μL | 2 μL | 1 μL |

Second-round PCR was performed as follows: 90 μL of ddH2O was added to the first-round PCR products, the mixture was diluted 10 times and was left to stand at the room temperature for 10 min after instantaneous centrifugation. A diluent of the first-round PCR products was used as an amplification template of the second-round PCR. An amplification system and components are shown in Table 4.3.

A PCR procedure was performed as follows: pre-denaturation was performed at 95° C. for 15 min; denaturation was performed at 94° C. for 30 s, annealing was performed at 60° C. for 4 min, extension was performed at 72° C. for 30 s, and 5 cycles were performed; denaturation was performed at 94° C. for 30 s, annealing was performed at 65° C. for 1 min, extension was performed at 72° C. for 30 s, and 10 cycles were performed.

TABLE 4.3
Construction of Second-round PCR Amplification
System of Targeted Re-sequencing Libraries

| | | Buffer | Barcode | dNTP | Taq | Sam- | Mg²⁺ |
|---|---|---|---|---|---|---|---|
| | ddH₂O | 10× | 2 uM | 2.5 mM | 5 U/μL | ple | 100 mM |
| 20 μL | 2.5 μL | 2 μL | 3.6 μL | 0.8 μL | 0.1 μL | 10 μL | 1 μL |

After PCR amplification ended, all the PCR products were subjected to 3% agarose gel electrophoresis detection and qRT-PCR quality control, all the qualified second-round PCR amplification products were mixed, and sequencing libraries were constructed. The sequencing libraries were sequenced, according to a 150 bp paired-end sequencing strategy, on the IlluminaHiSeqX-ten sequencing platform. Original data of about 5.79 Gb were finally obtained, including 38.6 million paired-end reads of 150 bp, and the average sequencing depth was 8,102×. The average Q30 and GC contents of sequencing data of all the samples were about 93% and about 48% respectively.

3. Filtering of data and screening of variations

After the sequencing reads of the various samples were classified according to barcode sequence information, reads containing more than 10% of poly-N sequences, low-quality reads (with more than 50% of base Phred scores lower than 5%) and reads containing wrong barcode sequences were removed by means of SOAPnuke software. Clean reads (36.48 M reads) of about 5.39 Gb were obtained for subsequent analysis.

These high-quality paired-end sequencing reads were compared with the zbtb38 reference gene sequence of Ictalurus punctatus by means of SOAP2.22 software, and the average comparison rate of the 129 samples was 93.86%. On the basis of SOAP comparison results, SNPs and Indel (Insertion deletion) variation information were retrieved from all the sequencing samples by means of SOAPsnp v1.05 and SOAPindel (v2.1) respectively. In order to ensure the accuracy of typing of the SNP molecular markers, the samples with the comparison rate lower than 80% were removed. According to statistics, 16 SNP molecular markers in total were identified in the zbtb38 gene coding region of Ictalurus punctatus, and no InDel markers were detected in all the samples. The 16 SNP molecular markers were located at bases 183, 366, 431, 733, 953, 1,102, 1,107, 1,855, 2,073, 2,409, 2,544, 2,825, 2,836, 3,070, 3,177 and 3,375 of the zbtb38 gene coding region, and respectively named g.zbtb38cds 183 C>T, g.zbtb38cds 366 G>A, g.zbtb38cds 431 G>A, g.zbtb38cds 733 G>A, g.zbtb38cds 953 A>C, g.zbtb38cds 1102 G>A, g.zbtb38cds 1107 T>A, g.zbtb38cds 1855 G>C, g.zbtb38cds 2073 T>C, g.zbtb38cds 2409 T>C, g.zbtb38cds 2544 G>A, g.zbtb38cds 2825 T>C, g.zbtb38cds 2836 C>T, g.zbtb38cds 3070 A>G, g.zbtb38cds 3177 T>C and g.zbtb38cds 3375 A>G.

4. Identification of sex-linked SNP molecular markers

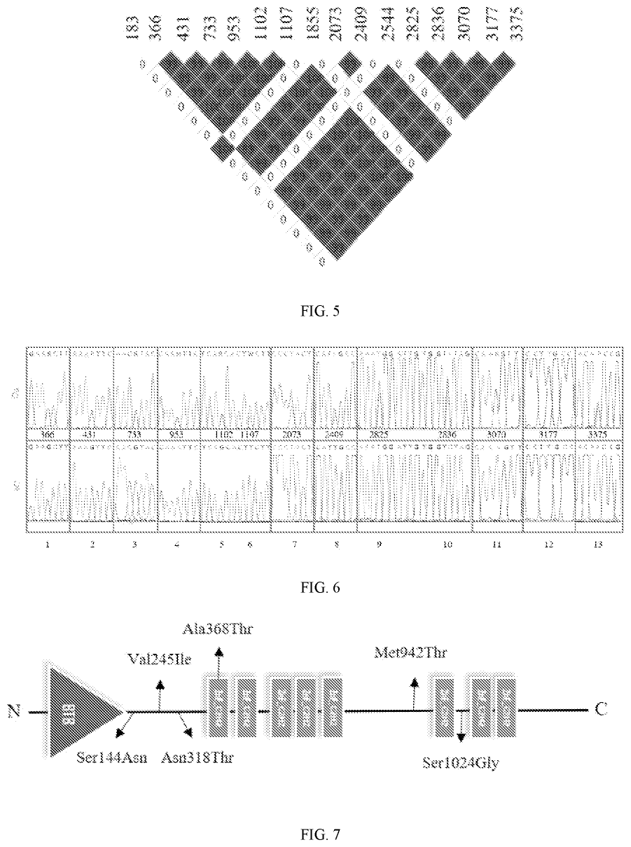

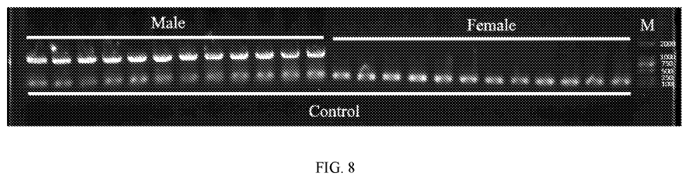

Linkage analysis of all the SNP sites was performed by means of the SHEsis software, and the sex-linked SNP molecular markers of Ictalurus punctatus were determined according to the linkage analysis results. The results are shown in FIG. 5. Linkage disequilibrium analysis showed that 13 of the 16 SNP molecular markers were sex-linked SNP molecular markers, and the other 3 were internal variations of X chromosome. The 13 sex-linked SNP molecular markers were located at bases 366, 431, 733, 953, 1,102, 1,107, 2,073, 2,409, 2,825, 2,836, 3,070, 3,177 and 3,375 of the zbtb38 gene coding region, and respectively named g. zbtb38cds 366 G>A, g. zbtb38cds 431 G>A, g. zbtb38cds 733 G>A, g. zbtb38cds 953 A>C, g. zbtb38cds 1102 G>A, g. zbtb38cds 1107 T>A, g. zbtb38cds 2073 T>C, g. zbtb38cds 2409 T>C, g. zbtb38cds 2825 T>C, g. zbtb38cds 2836 C>T, g. zbtb38cds 3070 A>G, g.zbtb38cds 3177 T>C and g.zbtb38cds 3375 A>G. The sequencing peak diagram is shown in FIG. 6. The genotypes of the 13 sex-linked SNP molecular markers in the female and male individuals are shown in Table 5. The markers were of heterozygous genotypes in all the male individuals and were of homozygous genotypes in all the female individuals, indicating that a sex determination system of Ictalurus punctatus may be the XX/XY type and the males were heterogamete. Among the 13 SNPs of the zbtb38 gene coding region, six substitutions resulted in changes in amino acids encoded by zbtb38-X and zbtb38-Y genes respectively (FIG. 7; Table 4.4), namely er144Asn, Va1245Ile, Asn318Thr, Ala368Thr, Met942Thr and Ser1024Gly, wherein Ala368Thr was located in the first zinc finger domain, and the other seven single-base substitutions did not cause changes in amino acid coding. On the basis of the above facts, zbtb38-Y genes were deemed as male-specific genes.

TABLE 4.4
Features of 13 Male Sex-linked SNP Markers in zbtb38 Gene Coding Region

| | | | | | | Amino | Amino |
|---|---|---|---|---|---|---|---|
| | | | | | | Acid | Acid |
| | | | | | | Encoded | Encoded |
| | No. | Reference | Variant | Female | Male | by | by |
| No. of SNP | ofAmplicon | Base | Base | Genotype | Genotype | zbtb38-X | zbtb38-Y |
| g. zbtb38ycds366 | CYH_3 | G | A | G/G | G/A | Lys | Lys |
| g. zbtb38ycds431 | CYH_3 | G | A | G/G | G/A | Ser | Asn |
| g. zbtb38ycds733 | CYH_5 | G | A | G/G | G/A | Val | Ile |
| g. zbtb38ycds953 | CYH_6 | A | C | A/A | A/C | Asn | Thr |
| g. zbtb38ycds1102 | CYH_7 | G | A | G/G | G/A | Ala | Thr |
| g. zbtb38ycds1107 | CYH_7 | T | A | T/T | T/A | Leu | Leu |
| g. zbtb38ycds2073 | CYH_13 | T | C | T/T | T/C | Pro | Pro |
| g. zbtb38ycds2409 | CYH_14 | T | C | T/T | T/C | Ile | Ile |
| g. zbtb38ycds2825 | CYH_17 | T | C | T/T | T/C | Met | Thr |
| g. zbtb38ycds2836 | CYH_17 | C | T | C/C | C/T | Leu | Leu |
| g. zbtb38ycds3070 | CYH_18 | A | G | A/A | A/G | Ser | Gly |
| g. zbtb38ycds3177 | CYH_19 | T | C | T/T | T/C | Leu | Leu |
| g. zbtb38ycds3375 | CYH_20 | A | G | A/A | A/G | Gln | Gln |

Embodiment 5: a genetic sex identification method based on SNP molecular markers

1. Design of primers was performed as follows: a pair of male-specific amplification primers CCMSF/R was designed according to locations of sex-linked SNP molecular markers, wherein the first to last base (g. zbtby38cds 1107 T>A) and the sixth to last base (g. zbtb38ycds 1102 G>A) of the forward primer as well as the first to last base (g. zbtb38ycds 2073 T>C) of the reverse primer were male-specific bases. At the same time, a pair of control primers CCCSF/R was designed in a region without mutation sites. The sequences of the primers are shown in Table 4.5.

TABLE 4.5
Genetic Sex Identification
Primers of Ictalurus punctatus

| Name of | | |
|---|---|---|
| Primer | Sequence of Primer (5′-3′) | Purpose |
| CCMSF | CCTTCAGTAGTTCAA(1102) | Male-specific |
| | CACTA( 1107) | Amplification |
| | (SEQ ID NO: 45) | Primer |
| CCMSR | ATCCACTATGGTTATTAGTG(2073) | Primer |
| | (SEQ ID NO: 46) | |
| CCCSF | TTATCAGACCCGTTGGAAGC | ControlPrimer |
| | (SEQ ID NO: 60) | |
| CCCSR | CTCCTGTGTGCCAGATCTCA | |
| | (SEQ ID NO: 61) | |

2. PCR amplification was performed as follows: 24 Ictalurus punctatus individuals with known sexes (12 males and 12 females) were collected, and genomic DNA was extracted. A double PCR amplification system was established by means of the male-specific amplification primers (SEQ ID NO: 45 and SEQ ID NO: 46) and the control primers (SEQ ID NO: 60 and SEQ ID NO: 61) to amplify male-specific bands and control bands at the same time. The 2× Taq Plus Master Mix (Dye Plus) kit (Vazyme Biotech Co., Ltd) was used for PCR amplification, and had a reaction system of 2× Taq Master Mix of 10 μL, the genomic DNA of 1 μL, each 10 μM primer of 0.5 μL, and ddH2O of 7 μL. Amplification conditions were as follows: pre-denaturation was performed at 94° C. for 5 min; denaturation was performed at 94° C. for 30 s, annealing was performed at 60° C. to 51° C. (1° C. lowered each cycle) for 30 s, extension was performed at 72° C. for 60 s, and 2 cycles were performed at each annealing temperature; denaturation was performed at 94° C. for 30 s, annealing was performed at 55° C. for 30 s, extension was performed at 72° C. for 60 s, and 15 cycles were performed; and extension was performed at 72° C. for 10 min.

3. Agarose gel electrophoresis was performed as follows: the PCR amplification products were subjected to electrophoresis by means of 1% agarose gel. After electrophoresis, the gel was placed on an UV projection detector to observe electrophoresis effects and determine the number and size of amplified bands of each sample.

4. Result analysis was performed as follows: in the male individuals, the male-specific amplification primers could amplify bands of 1,005 bp, and the control primers could amplify bands of 220 bp; and in the female individuals, only the control primers could amplify bands. The results are shown in FIG. 8. According to the locations of the sex-linked SNP molecular markers, three base mismatches were artificially made in the male-specific amplification primers (SEQ ID NO: 45 and SEQ ID NO: 46). The male-specific amplification primers could not be annealed with the genomic DNA of the female individuals, so that the bands could not be amplified in the female individuals.

5. Verification of the genetic sex identification method based on the SNP molecular markers was performed as follows: 96 samples were randomly selected from the sample library of the genetics and breeding center of Ictalurus punctatus for sex identification, so as to verify the accuracy of the genetic sex identification method. After the amplification products were subjected to 1% agarose gel electrophoresis, genetic sex identification results of the selected samples were compared with sex information recorded in the database, and the results showed that the accuracy of the genetic sex identification method reached 100%.

The embodiment provides the brand-new sex identification method of Ictalurus punctatus. The sex-linked SNP molecular markers of the male fishes are developed, the primers are designed by means of the locations of the markers, efficient and scientific identification is performed, through PCR, on the sexes of Ictalurus punctatus to be tested, and the accuracy reaches 100%.

What is claimed is:

1. A single nucleotide polymorphism (SNP) molecular marker for a weight gain trait selection of Ictalurus punctatus, comprising at least one of following SNP molecular markers:
a first SNP molecular marker for the weight gain trait selection, wherein the first SNP molecular marker is located at base 14,657,971 of chromosome 20, a mutation type of the first SNP molecular marker is C/G, the first SNP molecular marker is named g. 20.14657971 C>T, and a nucleotide sequence of the first SNP molecular marker is shown in SEQ ID NO: 69;
a second SNP molecular marker for the weight gain trait selection, wherein the second SNP molecular marker is located at base 14,658,012 of chromosome 20, a mutation type of the second SNP molecular marker is C/T, the second SNP molecular marker is named g. 20. 14658012 C>G, and a nucleotide sequence of the second SNP molecular marker is shown in SEQ ID NO: 62;
a first sex-linked SNP molecular marker based on zbtb38-Y, wherein the first sex-linked SNP molecular marker is located at base 366 of a zbtb38-Y gene coding region, a mutation type of the first sex-linked SNP molecular marker is G/A, the first sex-linked SNP molecular marker is named g.zbtb38ycds 366 G>A, and a nucleotide sequence of the first sex-linked SNP molecular marker is shown in SEQ ID NO: 47;
a second sex-linked SNP molecular marker based on zbtb38-Y, wherein the second sex-linked SNP molecular marker is located at base 431 of the zbtb38-Y gene coding region, a mutation type of the second sex-linked SNP molecular marker is G/A, the second sex-linked SNP molecular marker is named g.zbtb38ycds 431 G>A, and a nucleotide sequence of the second sex-linked SNP molecular marker is shown in SEQ ID NO: 48;
a third sex-linked SNP molecular marker based on zbtb38-Y, wherein the third sex-linked SNP molecular marker is located at base 733 of the zbtb38-Y gene coding region, a mutation type of the third sex-linked SNP molecular marker is G/A, the third sex-linked SNP molecular marker is named g.zbtb38ycds 733 G>A, and a nucleotide sequence of the third sex-linked SNP molecular marker is shown in SEQ ID NO: 49;
a fourth sex-linked SNP molecular marker based on zbtb38-Y, wherein the fourth sex-linked SNP molecular marker is located at base 953 of the zbtb38-Y gene coding region, a mutation type of the fourth sex-linked SNP molecular marker is A/C, the fourth sex-linked SNP molecular marker is named g.zbtb38ycds 953 A>C, and a nucleotide sequence of the fourth sex-linked SNP molecular marker is shown in SEQ ID NO: 50;
a fifth sex-linked SNP molecular marker based on zbtb38-Y, wherein the fifth sex-linked SNP molecular marker is located at base 1,102 of the zbtb38-Y gene coding region, a mutation type of the fifth sex-linked SNP molecular marker is G/A, the fifth sex-linked SNP molecular marker is named g.zbtb38ycds 1102 G>A, and a nucleotide sequence of the fifth sex-linked SNP molecular marker is shown in SEQ ID NO: 51;
a sixth sex-linked SNP molecular marker based on zbtb38-Y, wherein the sixth sex-linked SNP molecular marker is located at base 1,107 of the zbtb38-Y gene coding region, a mutation type of the sixth sex-linked SNP molecular marker is T/A, the sixth sex-linked SNP molecular marker is named g.zbtb38ycds 1107 T>A, and a nucleotide sequence of the sixth sex-linked SNP molecular marker is shown in SEQ ID NO: 52;
a seventh sex-linked SNP molecular marker based on zbtb38-Y, wherein the seventh sex-linked SNP molecular marker is located at base 2,073 of the zbtb38-Y gene coding region, a mutation type of the seventh sex-linked SNP molecular marker is T/C, the seventh sex-linked SNP molecular marker is named g.zbtb38ycds 2073 T>C, and a nucleotide sequence of the seventh sex-linked SNP molecular marker is shown in SEQ ID NO: 53;
an eighth sex-linked SNP molecular marker based on zbtb38-Y, wherein the eighth sex-linked SNP molecular marker is located at base 2,409 of the zbtb38-Y gene coding region, a mutation type of the eighth sex-linked SNP molecular marker is T/C, the eighth sex-linked SNP molecular marker is named g.zbtb38ycds 2409 T>C, and a nucleotide sequence of the eighth sex-linked SNP molecular marker is shown in SEQ ID NO: 54;
a ninth sex-linked SNP molecular marker based on zbtb38-Y, wherein the ninth sex-linked SNP molecular marker is located at base 2,825 of the zbtb38-Y gene coding region, a mutation type of the ninth sex-linked SNP molecular marker is T/C, the ninth sex-linked SNP molecular marker is named g.zbtb38ycds 2825 T>C, and a nucleotide sequence of the ninth sex-linked SNP molecular marker is shown in SEQ ID NO: 55;
a tenth sex-linked SNP molecular marker based on zbtb38-Y, wherein the tenth sex-linked SNP molecular marker is located at base 2,836 of the zbtb38-Y gene coding region, a mutation type of the tenth sex-linked SNP molecular marker is C/T, the tenth sex-linked SNP molecular marker is named g.zbtb38ycds 2836 C>T, and a nucleotide sequence of the tenth sex-linked SNP molecular marker is shown in SEQ ID NO: 56;
an eleventh sex-linked SNP molecular marker based on zbtb38-Y, wherein the eleventh sex-linked SNP molecular marker is located at base 3,070 of the zbtb38-Y gene coding region, a mutation type of the eleventh sex-linked SNP molecular marker is A/G, the eleventh sex-linked SNP molecular marker is named g.zbtb38ycds 3070 A>G, and a nucleotide sequence of the eleventh sex-linked SNP molecular marker is shown in SEQ ID NO: 57;
a twelfth sex-linked SNP molecular marker based on zbtb38-Y, wherein the twelfth sex-linked SNP molecular marker is located at base 3,177 of the zbtb38-Y gene coding region, a mutation type of the twelfth sex-linked SNP molecular marker is T/C, the twelfth sex-linked SNP molecular marker is named g.zbtb38ycds 3177 T>C, and a nucleotide sequence of the twelfth sex-linked SNP molecular marker is shown in SEQ ID NO: 58; and
a thirteenth sex-linked SNP molecular marker based on zbtb38-Y, wherein the thirteenth sex-linked SNP molecular marker is located at base 3,375 of the zbtb38-Y gene coding region, a mutation type of the thirteenth sex-linked SNP molecular marker is A/G, the thirteenth sex-linked SNP molecular marker is named g.zbtb38ycds 3375 A>G, and a nucleotide sequence of the thirteenth sex-linked SNP molecular marker is shown in SEQ ID NO: 59.

2. A screening method of the SNP molecular marker for the weight gain trait selection of the Ictalurus punctatus according to claim 1, comprising the following steps:
1) extracting DNA of fish samples to be tested, constructing genome re-sequencing libraries of the Ictalurus punctatus, and performing PE150 paired-end sequencing on the genome re-sequencing libraries on a BGISEQ-500 sequencing platform;
2) obtaining a monthly weight gain of the fish samples to be tested;
3) performing sequencing data filtering and SNP typing: after filtering and quality control, original sequencing data are compared with a reference genome of the Ictalurus punctatus, and SNPs are detected by a GATK software, captured variations are annotated by an ANNOVAR software, and genetic locations of genetic variations are determined by a VCFtools software; and
4) performing a genome-wide association study (GWAS) of growth traits by a single site mixed linear model GWAS (EMMAX) of SNP & Variation Suit v8.5.0 software to obtain a target SNP molecular marker.

3. A method for rapidly detecting genetic sexes of the Ictalurus punctatus with the SNP molecular marker according to claim 1, comprising the following steps:
1) designing peripheral primers, allele-specific extension (ASE) primers and DNA probes for amplifying SNP sites for the genetic sex identification of the Ictalurus punctatus, wherein sequences of the peripheral primers, the ASE primers and the DNA probes are shown in SEQ ID NO: 63 to SEQ ID NO: 66, and last bases at 3′ ends of the ASE primers are male-specific bases of the Ictalurus punctatus;
2) marking 5′ ends of the ASE primers with FAM fluorophores, marking 3′ ends of probes ASE-DP with Biotin, and testing a relationship between secondary structures of the ASE primers and the probes ASE-DP by an Oligo software, so as to avoid primer dimers;
3) performing a polymerase chain reaction (PCR) amplification by DNA of fish samples to be tested and the peripheral primers in step 1) to obtain first-round PCR amplification products;
4) performing a second-round PCR extension reaction and a nucleic acid hybridization by the first-round PCR amplification products in step 3) and the ASE primers and the probes ASE-DP in step 1) to obtain second-round PCR amplification products and ASE-DP hybridization nucleic acid products; and
5) detecting the ASE-DP hybridization nucleic acid products by rapid nucleic acid test strips, and judging the genetic sexes of the fish samples to be tested according to color development results of the rapid nucleic acid test strips.

4. A method for screening sex-linked SNP molecular markers of Ictalurus punctatus on the basis of male-specific genes zbtb38-Y of the Ictalurus punctatus, comprising the following steps:
1) designing specific primers for zbtb38 gene coding regions of the Ictalurus punctatus, wherein sequences of the specific primers are shown in SEQ ID NO: 1 to SEQ ID NO: 44;
2) performing a first-round PCR amplification by genomic DNA of female and male Ictalurus punctatus and the specific primers to obtain first-round products;
3) performing a second-round PCR amplification by the first-round products in step 2) and a barcode to obtain second-round female and male amplification products;
4) constructing sequencing libraries after mixing the second-round female and male amplification products in step 3), wherein the sequencing libraries are subjected to 150 bp paired-end sequencing on an Illumina HiSeqX-ten sequencing platform; and
5) comparing original sequencing data with zbtb38 genes after filtering original sequencing data, and regarding SNP molecular markers, linked with sexes, in samples with a comparison rate greater than 80% as the sex-linked SNP molecular markers.

5. A method of using the SNP molecular marker for the genetic sex identification according to claim 1, comprising the following steps:
1) designing male-specific amplification primers for the sex-linked SNP molecular markers, wherein in the male-specific amplification primers, the first to last base (g.zbtb38ycds 1107 T>A) and the sixth to last base (g.zbtb38ycds 1102 G>A) of a forward primer sequence are male-specific bases, and the first to last base (g.zbtb38ycds 2073 T>C) of a reverse primer sequence is a male-specific base;
2) designing a pair of control primers in a region without mutation sites, wherein the control primers are configured to amplify bands in both female and male individuals; and
3) establishing a double PCR amplification reaction by the male-specific amplification primers and the control primers, and detecting PCR amplification products by a 1% agarose gel.

6. The method according to claim 5, wherein sequences of the male-specific amplification primers are shown in SEQ ID NO: 45 and SEQ ID NO: 46.

7. A primer for developing an SNP molecular marker for a trait selection of Ictalurus punctatus, wherein the primer comprises one or more of sequences shown in SEQ ID NO: 1 to SEQ ID NO: 69.


SEQUENCE LISTING (69 sequences)

SEQ ID NO: 1 - DNA, 21 bp, Artificial Sequence
  The sequence is synthetized.
atgatggtggtccattccagc

SEQ ID NO: 2 - DNA, 21 bp, Artificial Sequence
  The sequence is synthetized.
tcagaggctgtgaaagcgttg

SEQ ID NO: 3 - DNA, 22 bp, Artificial Sequence
  The sequence is synthetized.
gctgcgactagcggatatttcc

SEQ ID NO: 4 - DNA, 25 bp, Artificial Sequence
  The sequence is synthetized.
cagagtcttgcctttcaatctcaag

SEQ ID NO: 5 - DNA, 25 bp, Artificial Sequence
  The sequence is synthetized.
caacaagtccttaatagctgcaggg

SEQ ID NO: 6 - DNA, 28 bp, Artificial Sequence
  The sequence is synthetized.
tctgtgatggagaaagcattagttatcc

SEQ ID NO: 7 - DNA, 18 bp, Artificial Sequence
  The sequence is synthetized.
caccacggcttgaggagc

SEQ ID NO: 8 - DNA, 24 bp, Artificial Sequence
  The sequence is synthetized.
cagcgtatgagtgttcaaacatgg

SEQ ID NO: 9 - DNA, 23 bp, Artificial Sequence
  The sequence is synthetized.
cgctatcaggaataccctctgtc

SEQ ID NO: 10 - DNA, 25 bp, Artificial Sequence
  The sequence is synthetized.
actagtttcactggatatgggtgcc

SEQ ID NO: 11 - DNA, 24 bp, Artificial Sequence
  The sequence is synthetized.
gactctgtccaaccatagacttgg

SEQ ID NO: 12 - DNA, 20 bp, Artificial Sequence
  The sequence is synthetized.
aaggcggatcatcatcctgt

SEQ ID NO: 13 - DNA, 20 bp, Artificial Sequence
  The sequence is synthetized.
ccttgtgttctgcacccgac

SEQ ID NO: 14 - DNA, 25 bp, Artificial Sequence
  The sequence is synthetized.
gcagtacttgcaaaacagatgactg

SEQ ID NO: 15 - DNA, 24 bp, Artificial Sequence
  The sequence is synthetized.
ccatatgcaaagccacaagagaag

SEQ ID NO: 16 - DNA, 25 bp, Artificial Sequence
  The sequence is synthetized.
ggaggatcttgaagatcagaacttg

SEQ ID NO: 17 - DNA, 24 bp, Artificial Sequence
  The sequence is synthetized.
caagtagcacacccttcctagagg

SEQ ID NO: 18 - DNA, 28 bp, Artificial Sequence
  The sequence is synthetized.
ctttattgcagtagtgacaaggataggc

SEQ ID NO: 19 - DNA, 23 bp, Artificial Sequence
  The sequence is synthetized.
gacatgagaatgtgcattcatgg

SEQ ID NO: 20 - DNA, 24 bp, Artificial Sequence
  The sequence is synthetized.
agattttttattcactgccagacg

SEQ ID NO: 21 - DNA, 22 bp, Artificial Sequence
  The sequence is synthetized.
ccatcagaagtcatttcatggc

SEQ ID NO: 22 - DNA, 22 bp, Artificial Sequence
  The sequence is synthetized.
ctgtgcctttaagtggagcagc

SEQ ID NO: 23 - DNA, 23 bp, Artificial Sequence
  The sequence is synthetized.
actgccccgctagatgttaactc

SEQ ID NO: 24 - DNA, 28 bp, Artificial Sequence
  The sequence is synthetized.
gttaaagccttcctcttcgatatgagac

SEQ ID NO: 25 - DNA, 28 bp, Artificial Sequence
  The sequence is synthetized.
caacatgtagaaacttctttgccttctg

SEQ ID NO: 26 - DNA, 24 bp, Artificial Sequence
  The sequence is synthetized.
ggaatggcataacatctctgcttg

SEQ ID NO: 27 - DNA, 25 bp, Artificial Sequence
  The sequence is synthetized.
actcatctcccacacaatgcagtag

SEQ ID NO: 28 - DNA, 20 bp, Artificial Sequence
  The sequence is synthetized.
accaggacaagcaggcttgg

SEQ ID NO: 29 - DNA, 18 bp, Artificial Sequence
  The sequence is synthetized.
ctgacgccgaaccttcgc

SEQ ID NO: 30 - DNA, 28 bp, Artificial Sequence
  The sequence is synthetized.
gaatcctagatcatcctctgctgtgttc

SEQ ID NO: 31 - DNA, 27 bp, Artificial Sequence
  The sequence is synthetized.
agagaagaaactggaagcaagataatg

SEQ ID NO: 32 - DNA, 27 bp, Artificial Sequence
  The sequence is synthetized.
agagaagaaactggaagcaagataatg

SEQ ID NO: 33 - DNA, 22 bp, Artificial Sequence
  The sequence is synthetized.
ggcaaaagattcagatccaagc

SEQ ID NO: 34 - DNA, 22 bp, Artificial Sequence
  The sequence is synthetized.
gacaatatgcatgctgagggtg

SEQ ID NO: 35 - DNA, 22 bp, Artificial Sequence
  The sequence is synthetized.
ctgccattgctgtagtagctcc

SEQ ID NO: 36 - DNA, 20 bp, Artificial Sequence
  The sequence is synthetized.
cagtatgagcagcggtagcg

SEQ ID NO: 37 - DNA, 24 bp, Artificial Sequence
  The sequence is synthetized.
actcgatagggttgactcgtagcc

SEQ ID NO: 38 - DNA, 27 bp, Artificial Sequence
  The sequence is synthetized.
cttgtgattgtctcactattttctgtc

SEQ ID NO: 39 - DNA, 25 bp, Artificial Sequence
  The sequence is synthetized.
catgagagcaagcacttcaataagg

SEQ ID NO: 40 - DNA, 24 bp, Artificial Sequence
  The sequence is synthetized.
gtcctgtcacctttacaggacaac

SEQ ID NO: 41 - DNA, 23 bp, Artificial Sequence
  The sequence is synthetized.
ttatccccaaatccctcaactcc

SEQ ID NO: 42 - DNA, 29 bp, Artificial Sequence
  The sequence is synthetized.
catgttgtatagaggttggatgtgttgag

SEQ ID NO: 43 - DNA, 28 bp, Artificial Sequence
  The sequence is synthetized.
gctttgtggaatattctaaagactccac

SEQ ID NO: 44 - DNA, 24 bp, Artificial Sequence
  The sequence is synthetized.
tcagttgtgggcataaaaaacagg

SEQ ID NO: 45 - DNA, 20 bp, Artificial Sequence
  The sequence is synthetized.
ccttcagtagttcaacacta

SEQ ID NO: 46 - DNA, 20 bp, Artificial Sequence
  The sequence is synthetized.
atccactatggttattagtg

SEQ ID NO: 47 - DNA, 21 bp, Artificial Sequence
  The sequence is synthetized.
ttcttgagaarcttcttgaga

SEQ ID NO: 48 - DNA, 21 bp, Artificial Sequence
  The sequence is synthetized.
aaatcagaaarttcaatgaat

SEQ ID NO: 49 - DNA, 21 bp, Artificial Sequence
  The sequence is synthetized.
aaaagagaacrtacaacaata

SEQ ID NO: 50 - DNA, 21 bp, Artificial Sequence
  The sequence is synthetized.
tcatgctcaamttcagccttg

SEQ ID NO: 51 - DNA, 21 bp, Artificial Sequence
  The sequence is synthetized.
ggccttcagtagttcarcact

SEQ ID NO: 52 - DNA, 21 bp, Artificial Sequence
  The sequence is synthetized.
cactwcttgctgtccatatgc

SEQ ID NO: 53 - DNA, 21 bp, Artificial Sequence
  The sequence is synthetized.
gctttaacccyactaataacc

SEQ ID NO: 54 - DNA, 21 bp, Artificial Sequence
  The sequence is synthetized.
aaacatacatygccaagcctg

SEQ ID NO: 55 - DNA, 21 bp, Artificial Sequence
  The sequence is synthetized.
ttcatggaaayggattgtggt

SEQ ID NO: 56 - DNA, 21 bp, Artificial Sequence
  The sequence is synthetized.
ggattgtggtytagaggaaag

SEQ ID NO: 57 - DNA, 21 bp, Artificial Sequence
  The sequence is synthetized.
tgcctgccaargttgcacaga

SEQ ID NO: 58 - DNA, 21 bp, Artificial Sequence
  The sequence is synthetized.
tcctctacctygccaccaaaa

SEQ ID NO: 59 - DNA, 21 bp, Artificial Sequence
  The sequence is synthetized.
gcaaagaacarccgaaaattg

SEQ ID NO: 60 - DNA, 20 bp, Artificial Sequence
  The sequence is synthetized.
ttatcagacccgttggaagc

SEQ ID NO: 61 - DNA, 20 bp, Artificial Sequence
  The sequence is synthetized.
ctcctgtgtgccagatctca

SEQ ID NO: 62 - DNA, 21 bp, Artificial Sequence
  The sequence is synthetized.
gttgaaaactytgatttttac

SEQ ID NO: 63 - DNA, 20 bp, Artificial Sequence
  The sequence is synthetized.
gaaatgtcttggctgcgact

SEQ ID NO: 64 - DNA, 20 bp, Artificial Sequence
  The sequence is synthetized.
ctcttgctcctcaagccgtg

SEQ ID NO: 65 - DNA, 17 bp, Artificial Sequence
  The sequence is synthetized.
tcccctttcttgagaaa

SEQ ID NO: 66 - DNA, 16 bp, Artificial Sequence
  The sequence is synthetized.
gcctttcaatctcaag

SEQ ID NO: 67 - DNA, 21 bp, Artificial Sequence
  The sequence is synthetized.
cgatatggacaccgacctgag

SEQ ID NO: 68 - DNA, 21 bp, Artificial Sequence
  The sequence is synthetized.
aggtaaagctcgactgtggtg

SEQ ID NO: 69 - DNA, 21 bp, Artificial Sequence
  The sequence is synthetized.
tactaactagsctctttaaaa